(19) United States
(12) Patent Application Publication
(10) Pub. No.: US 2025/0076279 A1
(43) Pub. Date: Mar. 6, 2025

(54) METHODS AND DEVICES FOR MANAGING INFORMATION DEALING WITH A MEDICAL TEST FOR ASSESSING A PREDETERMINED MEDICAL CONDITION

(71) Applicant: Roche Molecular Systems, Inc., Pleasanton, CA (US)

(72) Inventors: Patricia Blank, Rotkreuz (CH); Fabrice Jean Barthelemy Bouniort, Rotkruez (CH); Lorenzo Milo Franco Colombo, Basel (CH); Thomas Deufel, Penzberg (DE); Stefan ERB, Basel (CH); Mikael Gencay, Indianapolis, IN (US); Beverly Goede, Hayward, CA (US); Alison Kuchta, Sunol, CA (US); Asad Preuss-Dodhy, Diching (DE); Hans Peter Ramge, Rotkreuz (CH); Hans Martin Hendrick Schaefer, Basel (CH); Warren Young

(21) Appl. No.: 18/250,961

(22) PCT Filed: Oct. 28, 2021
(86) PCT No.: PCT/EP2021/079931

§ 371 Date: Apr. 27, 2023

(30) Foreign Application Priority Data
Oct. 29, 2020 (EP) 20204734.6
Oct. 28, 2021 (WO) PCT/EP2021/079931

(51) Int. Cl. G01N 33/487; G16H 10/40; G16H 50/80

(52) U.S. Cl. CPC G01N 33/48792; G16H 10/40; G16H 50/80

(57) ABSTRACT

The invention relates to a method for managing information dealing with a medical test for assessing a predetermined medical condition by means of a distributed system (100) comprising a plurality of communication devices (210, 312, 322, 410), the method comprising obtaining by a user communication device (210) information on an access code provided in a test-kit for assessing a predetermined medical condition, the user communication device (210) being configured for receiving and displaying messages addressed to a unique identifier. Further, the method comprises providing instructions relating to analysis of the medical sample. A negative analysis result is communicated to the user communication device (210) of the user. In case of a positive analysis result, consultation by a medical entity is established. Further aspects relate to a user communication device, a computer program product, a central server and a medical test-kit for use with the proposed method.

### TECHNICAL FIELD

The present invention relates to methods for managing information dealing with a medical test for assessing a predetermined medical condition, wherein a medical test-kit is provided. Further aspects relate to a user communication device, a computer program product, a central server and a medical test-kit for use with the proposed method.

### BACKGROUND

Medical self-tests exist for a number of diseases and other medical conditions, which may be used by individuals to easily and anonymously obtain a reliable diagnosis. These self-tests, in particular self-tests relating to infectious diseases, may provide an important contribution to public healthcare. For example, the WHO encouraged as early as 2016 the implementation of self-testing to augment existing HIV (human immunodeficiency virus) testing services. As a result, 59 countries worldwide have implemented policies on HIV self-testing, while 53 other countries are currently developing such policies. Currently, the initial diagnosis of HIV implies several hurdles. Predominantly shame and fear of stigma, cumbersome access, denial, and time investments are the strongest drivers for a low testing rate, backing the fact that even at this stage in time, 25% of HIV-positive patients are undiagnosed. Similar problems apply in particular to other STDIs (sexually transmitted diseases).

Acceptability of, for example, rapid HIV self-testing is high, but potential users remain concerned about correct use, interpretation of test results, and linkage to care. In order to assist users in performing a medical self-test, it is known to provide software in the form of “apps” which may be executed on a user device such as a smart phone, tablet or a general-purpose computer. By means of such “apps”, the respective self-test can be supported by a third party, such as a medical entity, rendering the self-test to become supported medical self-tests.

The article “SMARTtest: A Smartphone App to Facilitate HIV and Syphilis Self- and Partner-Testing, Interpretation of Results, and Linkage to Care” by Iván C Balán et al., AIDS Behav. 2020 May; 24 (5): 1560-1573, describes an app to support self- and partner-testing. The app provides video and pictorial step-by-step instructions as well as sample test results presented textually (“positive”, “negative”). The app is further designed to provide HIV/syphilis status awareness and disclosure, and linkage to care.

A problem of the known methods and apps for assisting in self-testing, in particular relating to infectious diseases such as HIV, is that they address mainly the step of communicating the test result. They do not offer appropriate assistance to the user in case a test result is positive. In particular, the known methods and apps cannot ensure that the user receives appropriate treatment for his/her medical condition. In addition, in case a test result is negative, the known methods and apps cannot provide further assistance relating to prophylaxis and/or further testing.

It is thus an object of the present invention to provide integrated methods and corresponding systems that ensures that a user of a medical self-test receives any required support and guidance according to best medical practice.

A further problem of medical self-testing, or medical testing in general, is the problem of under-testing. A large number of high-risk patients do not adhere to the recommended testing frequency. Further, social stigma as well as the fear of a positive test result may prevent many patients from testing using the known testing procedures.

It is thus a further object of the present invention to ensure the anonymity and privacy of a patient while at the same time enabling the patient to receive appropriate guidance and counseling from qualified medical practitioners by providing improved methods and systems for managing information relating to a medical test.

Yet another problem of known methods for assisting in testing is long turnaround time. Especially if a confirmatory test using a second analysis method is required in order to verify a first, positive test result, turnaround times are increased. Having to wait for a long time is a heavy burden for the patient, in particular in case of testing for severe medical conditions.

It is thus a further object of the present invention to provide methods and systems to assist in the communication of the involved entities such that an integrated testing process is provided especially in case a second analysis method is required for verification of a first, positive test result.

### SUMMARY OF THE INVENTION

A method for managing information dealing with a medical test for assessing a predetermined medical condition is proposed. The method comprises providing of a medical test-kit, the medical test-kit comprising an access code and a sampling unit for obtaining a medical sample, and the method comprises the further steps of:
by a user, inputting the access code in a user communication device, wherein the access code is verified, and after verification, is used as a unique identifier or a unique identifier is generated, and the user communication device being configured for receiving messages addressed to the respective unique identifier,
by the user, mailing of a medical sample labeled with a sample identifier associated to the unique identifier to a laboratory entity for analysis, the medical sample having been obtained using components of the sampling unit,
by the laboratory entity, receiving the mailed medical sample and analyzing the received medical sample using a first analysis method,
by the laboratory entity, in case a result of the first analysis method is negative, by means of a laboratory communication device, sending a message addressed by using the unique identifier to the user communication device, the message informing the user about the negative test result, or, in case the result of the first analysis method is positive, perform further processing, wherein the further processing comprises
by the laboratory entity or by a further laboratory entity, analyzing the received medical sample using a second analysis method,
by the laboratory entity or by the further laboratory entity, in case a result of the second analysis method is negative, by means of the laboratory communication device or a further laboratory communication device sending a message addressed by using the unique identifier to the user communication device, the message informing the user about the negative test result, or, in case the result of the second analysis method is positive, establishing consultation by a medical entity, such as a medical doctor, wherein the establishing comprises
by the laboratory entity or the further laboratory entity, by means of the laboratory communication device or a further laboratory communication device, sending a message to a practitioner communication device of the medical entity, the message comprising an indication of the positive test result and the unique identifier,
by the medical entity, communicating with the user, wherein communication is established using the practitioner communication device which is configured to contact the user communication device identified by means of the unique identifier.

The proposed method makes use of a distributed communication system comprising the user communication device, the laboratory communication device, if present the further the laboratory communication device, and the further practitioner communication device. This distributed communication system protects the privacy of the user of the medical test as the user communication device is only addresses by means of the unique identifier. This unique identifier is not associated with any further personal data and does not allow revealing of the user's identity.

The distributed communication system comprising a user communication device, a laboratory communication device, optionally a further laboratory communication device and a practitioner communication device, implements a method for managing information dealing with a medical test for assessing a predetermined medical condition, the method comprising the steps of:
obtaining by the user communication device information on an access code provided in a test-kit for assessing a predetermined medical condition, the user communication device being configured to verify the access code and for receiving and displaying messages addressed to a unique identifier, wherein the unique identifier is generated or the access code is used as unique identifier,
providing suggestions to a user to assist in obtaining a medical sample using components of a sampling unit provided in the test-kit on a display of the user communication device,
providing instructions to the user for mailing the medical sample obtained to a laboratory entity for analysis on the display of the user communication device, the medical sample being labeled with a sample identifier associated to the unique identifier,
by the laboratory communication device, obtaining the unique identifier associated with the mailed medical sample and providing instructions to a laboratory entity to perform a first analysis of the medical sample,
obtaining a result of a first analysis of the medical sample by the laboratory communication device, and, if the result is negative with respect to the predetermined medical condition, sending a message about the negative test result to the user communication device by means of the unique identifier, and, if the test result is positive with respect to the predetermined medical condition, providing instructions associated with the unique identifier to the laboratory entity or to a further laboratory entity to perform a second analysis of the medical sample using a second analysis method,
if second analysis has been performed, obtaining a result of the second analysis of the medical sample by the laboratory communication device or a further laboratory communication device, and, if the test result is negative with respect to the predetermined medical condition, sending a message about the negative test result to the user communication device by means of the unique identifier, and, if the test result is positive with respect to the predetermined medical condition, sending a message comprising the unique identifier to a practitioner communication device for establishing consultation by a medical entity, such as a medical doctor.

In conjunction with the described method, the user communication device is configured to receive messages sent to the user communication device and addressed by means of the unique identifier. In particular, the user communication device is configured to receive a message indicating that the medical sample has been received and to receive a message indicating a negative test result. Further, the user communication device is configured to alert the user that a message has been received and can be configured to display the contents of the message.

According to a specific embodiment, the user communication device, the laboratory communication device, if present the further laboratory communication device and/or the further practitioner communication device is a smart device such as a smartphone or a tablet, or is an internet enabled PC. Such a communication device is essentially a general purpose computer comprising a processor and memory, wherein the processor is configured to execute instructions of a computer program which may be stored in the memory. The respective communication devices, in particular the laboratory/further laboratory communication device and the practitioner communication device, may be part of existing information managing systems, such as electronic health record (EMR) systems in a doctor's office or hospital or laboratory information systems.

Suitable smart devices such as smartphones or tablets are well known and include in particular any smartphone or tablet running an iOS or Android operating system. Suitable internet enabled PCs include in particular any desktop or laptop PC running a common operating system such as Windows, Mac OS and Linux.

The respective computer program may be configured to cause the processor of the computer to execute the steps of the method which are to be carried out by the respective one of the communication devices. In this embodiment, a respective software application (“app”) is installed onto the respective communication device that is configured to carry out the steps of the method to be executed by the respective communication device. Such an “app” may be provided on a computer readable medium, such as a usb-pendrive, CD-ROM, DVD or Blu-ray Disc. Alternatively, such an “app” may be provided for download over a communication network such as the internet. The app may be stored in the memory of the respective communication device.

Alternatively, the respective “app” is configured as a “web-app” to be executed on a server, such as a webserver, and the communication device is configured to run a web browser or a terminal software that connects the communication device with the server. Such a server is essentially a computer comprising a processor and memory, wherein the processor is configured to execute instructions of a computer program. The computer program executed on the server causes the processor of the server to execute the respective steps of the method to be carried out by the respective communication device. The input and outputs are in this case relayed to the actual communication device of the respective entity which servers as front end for the application running on the server. For example, the user communication device may be a smartphone running a common web browser. For example, in order to execute the relevant steps of the method to be carried out by the user communication device, the user opens a provided website on his/her user communication device.

According to a specific embodiment, the respective communication device comprises output means, such as a display or a speaker, to present information. Further, the respective communication device can comprise input means to receive input such as the unique identifier. Suitable input means include, in particular, touch screens, keyboards, pointing devices such as a mouse, cameras, RFID readers, and microphones.

According to a specific embodiment, the user communication device, the laboratory communication device, if present the further the laboratory communication device, and the practitioner communication device are configured to communicate with a central server for relaying messages between said devices.

The respective communication devices and/or the central server can comprise communication means such as wired or wireless communication means, for connecting with a communication network, such as the internet.

Suitable wireless communication means include, communication modules configured for Wi-Fi, Bluetooth, GSM, UMTS, LTE, 5G or any other known wireless communication standard. Suitable wired communication means include communication modules such as modems or Ethernet interfaces.

The communication means can be configured to connect the respective communication device and/or the central server to a common communication network such as the internet.

The user may be any person or patient wishing to test for a certain medical condition such as a disease caused by a virus, a bacterium, a fungus or a parasite, including any an infectious disease. Examples for virus and bacteria causing diseases include human immunodeficiency virus (HIV), hepatitis B virus (HBV) and hepatitis C virus (HCV), human papillomaviruses (HPV), Chlamydia trachomatis, herpes simplex virus (HSV), Treponema pallidum and a coronavirus, such as severe acute respiratory syndrome coronavirus 2 (SARS-COV-2). The user may make use of a user communication device in order to send and receive communication dealing with the medical test.

The laboratory entity or the further laboratory entity may be any institution and/or any person having appropriate training for performing analysis relating to a medical test, such as medical personal. Further the term “laboratory entity” as well as “further laboratory entity” means to encompass any medical personal and associated staff receiving and/or processing the test analysis results of the first and/or second analysis. Also, the term “laboratory entity” as well as “further laboratory entity” is meant to encompass any agent managing the process of obtaining the first/second analysis test result and inputting the respective analysis test result into the laboratory/further laboratory communication device. The laboratory entity and, if present, the further laboratory entity, may make use of a laboratory communication device/further laboratory communication device in order to send and receive communication dealing with the medical test.

The medical entity may be any institution and/or any person having the appropriate training for treating patients and/or providing medical guidance, such as a medical practitioner or medical doctor, including support staff of the medical practitioner or medical doctor such as a nurse. In case of an institution, the medical entity may, for example, be a doctor's office or a hospital including any of the respective personal/staff. The medical entity may make use of a practitioner communication device in order to send and receive communication dealing with the medical test.

The access code is used as an authorization or proof of purchase. Also, the access code may be a code or identifier number to identify the test-kit. According to a specific embodiment, the access code is unique. The access code may be verified either by the user communication device or may be sent to the central server for verification. Verification may, for example, be performed by comparing the access code with known valid access codes stored in a database or by applying a mathematical function to the access code.

The unique identifier is a code that is used to address the user communication device and in particular allows sending of messages to the user communication device if a step of the proposed method requires the user to be contacted and further allows association of the medical sample with the user. The unique identifier does not allow revealing of the identity user and thus allows the user to perform anonymous medical testing. If the access code is unique, the access code may also be used as unique identifier. If the access code is not used as unique identifier, the user communication device may generate a unique identifier and send the identifier to the central server or the central server may generate the unique identifier and send the unique identifier to the user communication device. According to a specific embodiment, the unique identifier is randomly generated, is selected from a database comprising pre-generated identifiers or is generated by means of a deterministic mathematical function.

In case the unique identifier is not identical to the access code, the central server may have a database in which the generated/chosen unique identifier is stored and associated with the access code.

The sample identifier is a code used to identify the obtained medical sample. The sample identifier may be identical to the access code. If the access code is used as unique identifier, the sample identifier is also identical to the unique identifier. In case the unique identifier differs from the access code and/or the sample identifier is different from the access code, the sample identifier is associated to the unique identifier. For example, such an association may be performed during verification of the access code by means of a database linking access codes and corresponding sample identifiers together. Accordingly, the associating step may in particular be performed by a central server communicating with the user communication device.

After the user has acquired a test-kit, he/she will usually first setup his/her user communication device for use in one of the described methods. This may in particular involve downloading and/or installing of an appropriate software application (“app”) on the user communication device or launching of a web-browser in order to access an appropriate website or web-application.

In order to facilitate the initial setup of the user communication device, the medical test may include a reference to a downloadable computer program/software application (“app”). Such a reference can be provided in form of a hyperlink. The hyperlink may be encoded in form of barcode, such as a 2D matrix code (such as a QR Code) which may be printed on a paper or card, or may be encoded in a RFID tag. In particular, the hyperlink may direct the user to an app directory or app store to select, download and install the app.

After initial setup of the user communication device, the user may launch the respective “app” in order to proceed.

The user communication device can be further configured to provide general information relating to the medical test and/or the medical condition. Such information may include texts, images and/or videos relating to the use of the medical test or the medical condition, such as an infectious disease.

According to a specific embodiment, the step of inputting the access code in the user communication device or the step of obtaining the access code by the user communication device comprises scanning of a 1D or 2D matrix code using a camera of the user communication device or scanning of an RFID tag. Examples of 2D matrix code include QR (quick response) codes.

The user communication device can provide the user with instructions to enter the access code. For example, the user communication device may show a video on a display device which explains how an access code provided in form of a barcode or a QR code should be scanned.

After the access code has been entered into the user communication device, the access code can be verified and, after a successful verification, the user communication device guides the user through the following steps of the method.

According to a specific embodiment, prior to the step of mailing a medical sample, the user communication device provides detailed instructions to the user, the instructions comprising text, images, audio and/or video. In particular, the detailed instruction may explain the use of the test-kit in order to obtain the medical sample.

Such text, images, audio and/or video instructions may be stored alongside with a computer program/software application implementing the respective method steps on the user communication device or may be provided in form of an internet link.

Further, the user communication device may present detailed instructions explaining how the obtained medical example should be mailed to the laboratory entity. This may include displaying of a mailing address on a display of the user communication device. Alternatively, the medical test may include a pre-prepared mailing label or a pre-labeled mailing package, such as a bag or box.

The sampling unit can include a self-sampling device or a sample carrier, such as a plasma separation card or swab and/or bags or vessels for collecting of the medical-sample. According to a specific embodiment, the sample carrier also includes a sample identifier, for example in the form of a barcode, a 2D matrix code or an RFID tag. This sample identifier may be identical or different to the access code and is either identical to the unique identifier or is associated with the unique identifier. The access code may be used for associating of the unique identifier with the sample identifier, for example by means of a database linking an access code to a corresponding sample identifier.

According to a specific embodiment, the sampling unit is configured so that sufficient material is collected as medical-sample so that two independent analysis methods may be used for analysis of the medical-sample. For example, the sampling unit may include two sample carriers or a single sample carrier may be configured for collection of sufficient material for performing the two independent analysis methods.

The laboratory entity will receive the medical-sample by mail and will begin processing after receipt of the medical-sample. According to a specific embodiment, the laboratory entity will not receive any information that allows revealing of the identity of the user, such as a mailing address or a phone number, but only the unique identifier and the sample identifier, if the sample identifier is different from the unique identifier.

After receipt of the medical sample, the laboratory entity can enter the sample identification and/or the unique identifier upon receipt into the laboratory communication device, for example by scanning of a barcode, a 2D matrix code or an RFID tag. In case the sample identifier is not identical to the unique identifier, the laboratory communication device may obtain the unique identifier from a central server having a database linking the unique identifier to the sample identifier.

According to a specific embodiment, the method further comprises, by the laboratory entity, sending of a message addressed using the unique identifier to the user communication device, informing the user about receipt of the sample and/or testing of the sample and/or that testing has been completed. For example, a laboratory communication device may automatically generate and sent such a message after a bar code associated with the received medical sample has been scanned. This allows the user to stay up-to-date and makes the entire process as transparent as possible.

Upon receipt of the medical sample, the laboratory entity begins analysis of the medical sample using the first analysis method.

According to a specific embodiment, the first analysis method detects a protein, such as an antibody and/or an antigen. According to a specific embodiment, the second analysis method detects a nucleic acid, such as DNA or RNA. A suitable method for detection of nucleic acids is or includes polymerase chain reaction (PCR) and reverse transcription polymerase chain reaction (RT-PCR).

According to a specific embodiment, the first analysis method provides a quick result, can be performed easily, is inexpensive and can be carried out in large quantities. For the first analysis method, the occurrence of some false positive test results is acceptable as a second, different analysis method may be employed to confirm a positive test result. An antibody or antigen test delivers the test result typically in 10 to 20 minutes, is relatively cheap and capable of processing a large amount of samples so that such a test is particularly suitable as first analysis method.

After the first analysis has been completed, the test result can be entered into the laboratory communication device. According to a specific embodiment, an information system of the laboratory entity, which may be coupled to machines used in conjunction with performing of the first analysis, and the laboratory communication device are networked together so that the analysis result is automatically entered into the laboratory communication device. In such an embodiment, the laboratory communication device may be part of an existing laboratory information system. For example, an existing computer system may be configured to execute the steps of the laboratory communication device by providing appropriate software. Alternatively, the analysis result is manually entered into the laboratory communication device. In order to assist the staff of the laboratory entity in this task, the laboratory communication device may in particular be configured to scan a result encoded in a barcode.

If the test result of the first analysis method is positive, a further test using a second analysis method is performed as confirmatory test either by the same laboratory entity or a different laboratory entity. If the second analysis method is performed by a different, further laboratory entity, the laboratory communication device can provide the laboratory entity with instructions such as a mailing address in order to organize transport of the medical sample to the further laboratory entity. Additionally, the laboratory communication device may send a message comprising the unique identifier to a further laboratory communication device to announce the necessity of a second analysis to the further laboratory entity. According to a specific embodiment, the same sample carrier used for performing the first analysis method is passed on in order to perform the second analysis. Advantageously, this avoids the time required to obtain a second medical sample from the patient and significantly reduces turnaround times.

After receipt of the medical sample, the further laboratory entity can enter the sample identification and/or the unique identifier upon receipt into the further laboratory communication device, for example by scanning of a barcode, a 2D matrix code or an RFID tag.

Upon receipt of the medical sample, the further laboratory entity begins analysis of the medical sample using the second analysis method.

In case the second analysis method is performed by the same laboratory entity as the first analysis, the respective laboratory entity may immediately begin analysis of the medical sample using the second analysis method.

Advantageously, the second analysis method provides a high degree of certainty and thus a low percentage of false positive test results. As the second analysis method is only used if the first analysis method yielded a positive test result, it is acceptable that the second analysis method is slower and more expensive. PCR (polymerase chain reaction) or RT-PCR (reverse transcription polymerase chain reaction) has a very low false positive rate and is thus particularly suitable as second analysis method.

After the second analysis has been completed, the test result can be entered into the (further) laboratory communication device. According to a specific embodiment, an information system of the (further) laboratory entity, which may be coupled to machines used in conjunction with performing of the second analysis, and the further communication device are networked together so that the analysis result is automatically entered into the (further) laboratory communication device. Alternatively, the analysis result is manually entered into the (further) laboratory communication device. In order to assist the staff of the laboratory entity in this task, the (further) laboratory communication device may in particular be configured to scan a result encoded in a barcode.

The first and/or second analysis methods test for a medical condition, wherein the medical condition can correlate with a sexually transmitted disease and/or the medical condition can be a disease caused by a virus, a bacterium, a fungus or a parasite such as a virus or a bacterium selected from the group consisting of human immunodeficiency virus (HIV), hepatitis B virus (HBV) and hepatitis C virus (HCV), human papillomaviruses (HPV), Chlamydia trachomatis, herpes simplex virus (HSV), Treponema pallidum and a coronavirus, such as severe acute respiratory syndrome coronavirus 2 (SARS-COV-2).

If the first or the second analysis method yield a negative test result, the laboratory entity or the further laboratory entity can send an appropriate message to the user indicating the negative test result by means of the respective laboratory communication device and addressed by means of the unique identifier.

In case both analysis methods yield a positive test result, it is considered to be inappropriate to present the test result by means of a mere message to the user.

In particular, if the medical condition is related to an incurable condition or disease, a positive test result could cause distress in the user and the user should thus not be left alone. Accordingly, the proposed method will establish direct communication between a medical practitioner and the user to report the positive test result.

According to a specific embodiment, the user further provides a region code, e.g. a zip code, and the medical entity is selected based on the provided region code. Accordingly, the user communication device can be configured to obtain the region code from the user. Additionally, if several competent laboratory entities are available, the region code may be used to provide the user with an address of a laboratory close to his/her location in order to accelerate the process. According to a specific embodiment, the region code is only used in order to select a medical entity and/or a laboratory close to the location of the user and is not used for any other purposes. In particular, the region code may not be disclosed to any of the entities involved in the method such as the laboratory or the medical entity.

According to a specific embodiment, the step of communicating with the user comprises establishing a voice (telephone) and/or video link between the user communication device addressed by using the unique identifier and the further practitioner communication device. This voice and/or video link can be established using a software application “app” executed on the user communication device and the practitioner communication device so that no personalized address such as a phone number of the user is exposed. In particular, this functionality can be provided as an “in-app call”. The voice or video link may, for example, be established by means of voice-over-ip using a communication network such as the internet. Alternatively or additionally, communicating with the user may be performed in the form of a text chat. Advantageously, this allows the user to remain anonymous and at the same time allows for adequate medical counseling that is of particular importance when a positive test result and its implications are to be communicated to the user.

According to a specific embodiment, after establishing consultation by the medical entity, the method further comprises scheduling of an appointment of the user with the medical entity, wherein the unique identifier is transmitted by the user communication device to an appointment booking system of the medical entity for identification of the user.

For scheduling of an appointment, the practitioner communication device may interface with the appointment booking system of the medical entity and may receive one or more open dates for an appointment. The one or more open dates are then transmitted to the user communication device, wherein the unique identifier is again used to address the user communication device, and are presented to the user. The user may then select an appropriate date for booking and his/her response may then be sent to the appointment booking system via the practitioner communication device.

Alternatively, for scheduling of an appointment, the practitioner communication device may provide an internet link to the appointment booking system to the user communication device. The user may then open the provided link, for example using a web browser installed on his/her user communication device, and may then schedule an appointment.

The practitioner communication device may be provided as part of an existing electronic health record (EMR) system. For example, an existing computer system may be configured to execute the steps of the practitioner communication device by providing appropriate software.

According to a specific embodiment, the method further comprises, by the user communication device when a message indicating a negative test result has been received, the step of obtaining user input indicating whether the user belongs to a high risk group for the predetermined medical condition and, if the user belongs to a high risk group, performing one or more of
by the user communication device, generating a reminder for the user to repeat the test after a predetermined time has passed, and/or
by the user communication device, offering further testing in order to assess whether the user is eligible for a prophylaxis treatment relating to the predetermined medical condition.

For example, after performing a test for HIV, the user communication device may inquire from the user whether he/she would like his/her liver/renal function tested in order to assess whether they are sufficient to get PREP (pre exposition prophylaxis) prescribed. If so, the user communication device may present information relating to a further medical test for testing liver/renal function. In particular, such a medical test may assess creatinine and serum phosphate as biomarkers for liver and renal function, respectively.

In a further example, the user communication device may schedule an alert so that the user communication device alerts the user after a predetermined time has passed that the test should be repeated. In order to do so, a calendar function of the user communication device may be used. The predetermined time may be chosen in dependence on the respective medical condition and current medical recommendations.

The user communication device may further be configured to provide general health information, such as general information about STDIs (sexually transmitted diseases).

A further aspect of the invention is providing of a user communication device configured for use in one of the methods described herein, wherein the user communication device is configured to obtain an access code or unique identifier by means of user input, to verify the access code, and to generate a unique identifier or use the access code as unique identifier. The user communication device is further configured for receiving messages from a server addressed to said unique identifier.

Likewise, a further aspect of the invention is providing of a computer program comprising instructions, which, when executed by a computer, cause the computer to carry out the steps to be executed by the user communication device of one of the methods described herein, wherein the computer program includes instructions to cause the computer to obtain an access code by means of user input, generate a unique identifier or to use the access code as unique identifier, and to receive messages addressed to the unique identifier.

A further aspect of the invention is providing a laboratory communication device and optionally of a further laboratory communication device configured for use in one of the methods described herein.

Likewise, a further aspect of the invention is providing of a computer program comprising instructions, which, when executed by a computer, cause the computer to carry out the steps to be executed by the laboratory communication device of one of the methods described herein.

A further aspect of the invention is providing of a practitioner communication device configured for use in one of the methods described herein.

Likewise, a further aspect of the invention is providing of a computer program comprising instructions, which, when executed by a computer, cause the computer to carry out the steps to be executed by the practitioner communication device of one of the methods described herein.

The respective communication devices may be essentially configured as general purpose computer comprising a processor and memory, wherein the processor is configured to execute instructions of a computer program. The computer program may be stored in the memory of the respective communication device.

According to a specific embodiment, the respective communication device comprises output means, such as a display or a speaker, to present information. Further, the respective communication device can comprise input means to receive input. Suitable input means include, in particular, touch screens, keyboards, pointing devices such as a mouse, cameras, RFID readers, and microphones.

The respective communication devices comprise communication means such as wired or wireless communication means, for connecting with a communication network, such as the internet.

Suitable wireless communication means include, communication modules configured for Wi-Fi, Bluetooth, GSM, UMTS, LTE, 5G or any other known wireless communication standard. Suitable wired communication means include communication modules such as modems or Ethernet interfaces.

The communication means can be configured to connect the respective communication device to a common communication network such as the internet.

In order to implement the respective steps of the methods, the respective communication devices may comprise specialized modules. For example, the communication devices may comprise a messaging module handling sending and/or receiving of messages that are addressed by means of the unique identifier and relayed by the central server. Accordingly, the central server may also comprise modules for implementing certain steps of the methods, such as a messaging module for relaying of messages addressed by means of the unique identifier.

Another aspect of the invention is providing of a central server configured to carry out the steps of the central server of one of the methods described herein.

The central server is essentially a general purpose computer comprising a processor and memory, wherein the processor is configured to execute instructions of a computer program. The computer program may be stored in the memory of the central server. The central server may be configured as a dedicated machine implementing the method steps to be carried out by the central server, or may be configured as a virtual server or so called “cloud-server”, wherein one or more virtual servers share resources provided by one or more physical servers.

The central server comprises communication means such as wired or wireless communication means for connecting with a communication network, such as the internet.

The central server in conjunction with the user communication device, the laboratory communication device, if present the further laboratory communication device and the practitioner communication device form a distributed communication system capable of managing information relating to a medical test for assessing a predetermined medical condition. The components of the system are interconnected by means of a common communication network, such as the internet.

Advantageously, the provided system comprising the individual communication devices allows managing of the entire testing process and allow communication between the involved entities and the user without revealing the true identity of the user. Accordingly, the user's privacy is protected while ensuring smooth operation as well as providing the user with the required medical assistance and counseling. Still further, the system further assists in the follow up processes such as scheduling an appointment for further assistance by the medical practitioner as well as reminding the user to repeat the test at appropriate intervals and/or assists the user in further health programs such as prophylaxis.

From the user's perspective, he/she has only a single point of contact to the systems/methods provided by his/her user communication device. In case of a smart device such as a smartphone or tablet as user communication device, only a single software application (“app”) executed on the user communication device is sufficient to provide all the features and services to the user.

The user communication device (the software “app” to be executed on the user communication device) may advantageously provide the functions of a guidance app teaching the user how to perform self-sampling with the provided sampling unit, the functions of a medical information system about the respective medical condition, the functions of an appointment scheduling system and the functions of a result communication system.

A further aspect of the invention is providing of a medical test-kit comprising an access code, a reference for a downloadable computer program to be executed on a user communication device and a sampling unit for obtaining a medical sample, wherein the medical test-kit is configured to be used in the course of one of the methods described herein.

The sampling unit for obtaining a medical sample can be any suitable device including a self-sampling device and can comprise at least a sample carrier, such as a plasma separation card or swab. Further, the sampling unit can comprise one or more of disinfectant, tourniquet, lancet, capillaries, dispenser, small transparent sample bag, big sample bag, envelope and tape/plaster. In one embodiment the sample is or has been obtained by a non-invasive method, i.e. a method that does not infiltrate or destroy healthy tissue, such as a swab (e.g. vaginal or nasal), hair, tear, sweat, stool or urine sample.

In order to protect the contents of the test-kit from humidity and moisture, a bag with desiccant can be included.

In order to provide the user with an access code and/or a sample identifier that links the sampling unit to the user communication device and thus enables the communication method, the medical test-kit can include one or more code carrier in form of cards or stickers with barcodes, 2D matrix codes and/or RFID tags.

The access code may be different or identical to the sample identifier. In case the access code and the sample identifier are identical, the medical test-kit can comprise at least two identical code carriers. It is then intended that one of the code carriers is attached to the sample carrier in order to associate the sample carrier with the user, and at least one code carrier is intended to remain with the user in order to enable use of the user communication device. If the access code and the sample identifier are different, it is intended for a code carrier encoding the access code to remain with the user and the code carrier encoding the sample identifier is intended to be attached to the sample carrier.

The reference to the downloadable computer program can be provided in form of a hyperlink. The hyperlink may be encoded in form of barcode, such as a 2D matrix code (QR code), or may be encoded in a RFID tag. Accordingly, the medical test-kit comprises at least one code carrier encoding the hyperlink.

In addition to instructions offered by the downloadable computer program, the medical self-test-kit may of course also comprise written instructions, for example in the form of a printed booklet.

The envelope can be pre-labeled, and, additionally, the postage can already be paid so that the user can just mail the envelope after the sealed sample carrier marked with the sample identifier has been put into it.

According to a specific embodiment, the medical test-kit is appropriately packaged such that no special storage conditions such as a certain humidity and/or temperature range is required. Also, the package can be configured to provide sufficient protections against mechanical damage so that the medical test-kit may, for example, be sold in vending machines or may sent to the user by mail. Advantageously, this ensures the privacy of the user of the medical self-test from the beginning on.

The anonymity offered by the provided methods, systems and medical test-kit is important in order to overcome inhibitions of patients and thus in order to increase testing rates.

As used herein and also in the appended claims, the singular forms “a”, “an”, and “the” include plural reference unless the context clearly dictates otherwise. Similarly, the words “comprise”, “contain” and “encompass” are to be interpreted inclusively rather than exclusively; that is to say, in the sense of “including, but not limited to”. Similarly, the word “or” is intended to include “and” unless the context clearly indicates otherwise. The terms “plurality”, “multiple” or “multitude” refer to two or more, i.e. 2 or >2, with integer multiples, wherein the terms “single” or “sole” refer to one, i.e. =1. Furthermore, the term “at least one” is to be understood as one or more, i.e. 1 or >1, also with integer multiples. Accordingly, words using the singular or plural number also include the plural and singular number, respectively. Additionally, the words “herein,” “above,”, “previously” and “below” and words of similar import, when used in this application, shall refer to this application as a whole and not to any particular portions of the application.

To avoid repetition in the figures and the descriptions of the various aspects and illustrative embodiments, it should be understood that many features are common to many aspects and embodiments. The description of specific embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize. Specific elements of any foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure as defined by the appended claims. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for the reason of lucidity, if in a section of a drawing not all features of a part are provided with reference signs, it is referred to other sections of the same drawing. Like numbers in two or more figures represent the same or similar elements.

The following examples are intended to illustrate various specific embodiments of the present invention. As such, the specific modifications as discussed hereinafter are not to be construed as limitations on the scope of the present invention. It will be apparent to the person skilled in the art that various equivalents, changes, and modifications may be made without departing from the scope of the present invention, and it is thus to be understood that such equivalent embodiments are to be included herein. Further aspects and advantages of the present invention will become apparent from the following description of particular embodiments illustrated in the figures.

### BRIEF DESCRIPTION OF THE DRAWINGS

Accordingly, the invention is further described below on the basis of the drawings, in which:

FIG. 1 shows a schematic view of a distributed communication system,

FIG. 2 schematically depicts a user communication device,

FIG. 3 depicts a flow diagram of the method for managing information, and

FIG. 4 depicts a flow diagram of an embodiment of the method for managing information using a distributed communication system.

### LIST OF REFERENCE NUMERALS

100 distributed communication system 100
200 user's home
210 user communication device
211 processor
212 memory
213 communication means
214 output means
215 input means
310 first laboratory
312 laboratory communication device
320 second laboratory
322 further laboratory communication device
400 doctor's office
402 practitioner communication device
500 central server
1001 first step
1002 second step
1003 third step
1011 first result negative
1012 first result positive
1020 further processing step
1021 second result negative
1022 second result positive
1031 first result communication step
1032 second result communication step
1040 forwarding step
1041 first establishing step
1042 second establishing step
1043 third establishing step
2001 first step
2002 second step
2003 third step
2004 fourth step
2005 fifth step
2011 first result negative
2012 first result positive
2020 further processing step
2021 second result negative
2022 second result positive
2030 messaging step
2040 establishing step

### DETAILED DESCRIPTION

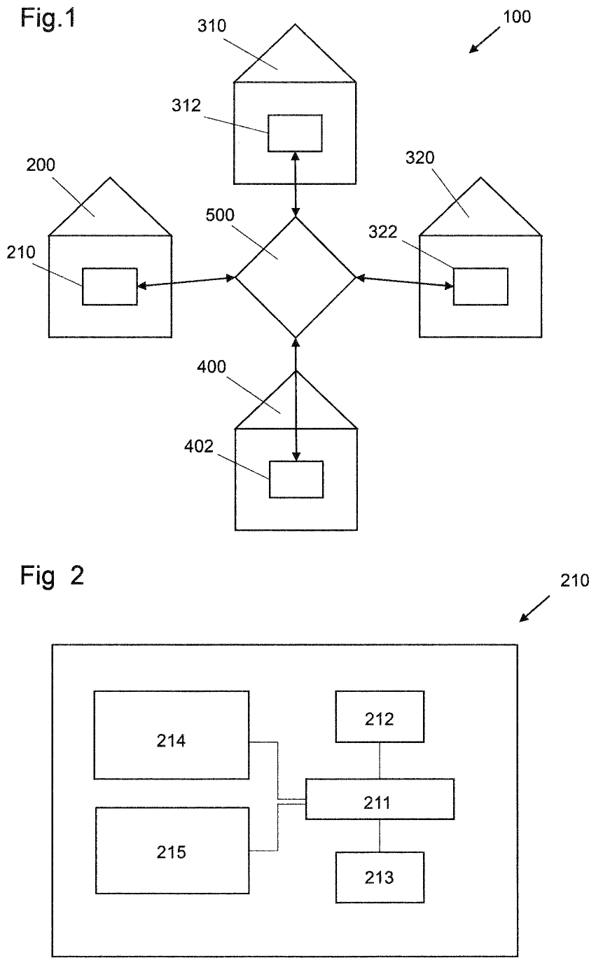

FIG. 1 schematically shows a distributed communication system 100. The distributed communication system 100 comprises a user communication device 210, a laboratory communication device 312, a further laboratory communication device 322, a practitioner communication device 402 and a central server 500.

The devices of the distributed communication system 100 are located and used at different places while being interconnected by means of the central server 500. The user communication device 210 is, for example, located at a home 200 of the user. The laboratory communication device 312 is, for example located at a first laboratory 310 performing the first analysis method according to one of the methods described herein. The further laboratory communication device 322 is, for example, located at a second laboratory 320 performing the second analysis method according to one of the methods described herein. The practitioner communication device 402 is, for example, located at a doctor's office 400.

FIG. 2 schematically depicts a user communication device 210. While FIG. 2 depicts an exemplary user communication device 210, the other communication devices of the distributed communication system 100, namely, the laboratory communication device 312, if present the further laboratory communication device 322 and the practitioner communication device 402 may have an identical setup and differ only in the loaded computer program or software application (“app”).

The exemplary user communication device 210 of FIG. 2 comprises a processor 211 connected to memory 212, communication means 213, output means 214 and input means 215. The communication means 213 can be configured to provide a wireless connection to a common communication network such as the internet for interconnecting all communication devices and the central server 500 of the distributed communication system 100. The memory 212 can store a computer program or software application (“app”) which causes the processor 211 to execute the steps to be carried out by the user communication device 210. The output means 214 can be configured as a display and/or a speaker. The input means 215 can include a camera for scanning of a bar code and can further comprise a touch screen input allowing the user to interact with the user communication device 210.

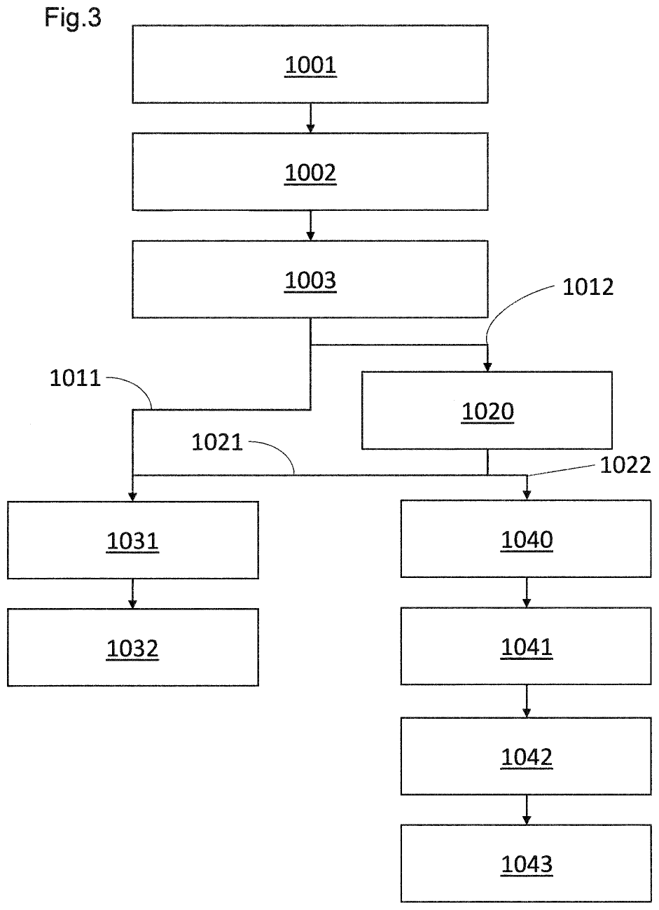

FIG. 3 depicts a flow diagram of an embodiment of the method for managing information relating to a medical test for assessing a predetermined medical condition. In this method, a medical test-kit is provided to a user who wishes to perform the medical test. The medical test-kit comprises an access code and a sampling unit for obtaining a medical sample. Further, the method relies on the use of communication devices by the respective entities. Such a communication device, may, for example, be provided in form of a general purpose computer which is connected to the internet or in form of a smart device such as a smartphone.

In a first step 1001 of the method, the user inputs the access code of the medical test-kit in a user communication device 210, see FIG. 1, wherein the access code is verified, and after verification, is used as a unique identifier or a unique identifier is generated. The user communication device 210 is configured for receiving messages addressed to the respective unique identifier, for example by means of an installed software application (“app”).

In a second step 1002 the user mails a medical sample labeled with a sample identifier associated to the unique identifier to a laboratory entity for analysis. The user communication device 210 may assist in this step, for example, by showing instruction on how to obtain the medical sample using components of the sampling unit.

In a third step 1003 of the method, the medical sample is received by the laboratory entity, receiving the mailed medical sample and the laboratory entity analyzes the received medical sample using a first analysis method.

In case of a first result of the first analysis method is negative, the user is informed about the negative test result as indicated by arrow 1011. In case the first result is positive with respect to the predetermined medical condition, further processing is performed as indicated by arrow 1012, wherein further processing comprises a further processing step 1020.

In the further processing step 1020, the medical sample is analyzed using a second analysis method either by the same laboratory entity or by a further laboratory entity. In case a further laboratory entity performs the further processing, the further processing step 1020 also comprises forwarding the medical sample to the further medical entity.

In case of a second result of the second analysis method is negative, the user is informed about the negative test result as indicated by arrow 1021. In case the second result is positive with respect to the predetermined medical condition, establishing consultation by a medical entity is initiated as indicated by arrow 1022.

Informing the user about the negative test result comprises a first result communication step 1031 in which a message indicating the negative result is sent to the user's communication device 210 which is addressed by means of the unique identifier by a laboratory communication device 312 of the laboratory entity or a further laboratory communication device 322 of the further laboratory entity, respectively. In a second result communication step 1032, the user communication device 210 displays the received message and thus communicates the negative test result to the user.

The establishing of consultation by a medical entity in case the second test result is positive comprises a forwarding step 1040 in which by means of a laboratory communication device 312 or a further laboratory communication device 322 a message is sent to a practitioner communication device 402, see FIG. 1, of the medical entity. The message comprises an indication of the positive test result and the unique identifier.

In a first establishing step 1041 the practitioner communication device 402 sends an invitation message to the user's communication device 210 which is addressed by means of the unique identifier. In a second establishing step 1042 the user communication device 210 displays the invitation and, in response to user input, may proceed to the third establishing step 1043 in which a call between the user and the medical entity is established, in particular in the form of an in-app call using voice-over-ip technology.

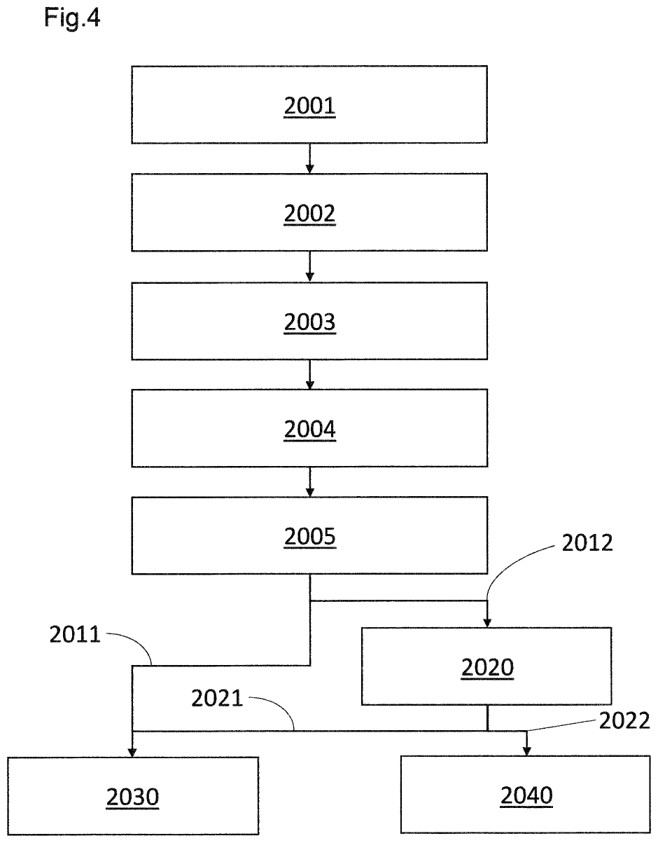

FIG. 4 depicts a flow diagram of an embodiment of the method for managing information using a distributed communication system 100 as shown in FIG. 1. The distributed communication system as described with respect to FIG. 1 comprises a user communication device 210, a laboratory communication device 312, optionally a further laboratory communication device 322 and a practitioner communication device 402. Further a medical test-kit is provided to a user who wishes to perform the medical test. The medical test-kit comprises an access code and a sampling unit for obtaining a medical sample.

In a first step 2001 of the method, the user communication device 210 obtains information on an access code provided in the test-kit for assessing a predetermined medical condition. The user communication device 210 is configured to verify the access code and for receiving and displaying messages addressed to a unique identifier, wherein the unique identifier is generated or the access code is used as unique identifier.

In a second step 2002, the user communication device 210 provides suggestions to a user in order to assist in obtaining a medical sample using components of the sampling unit provided in the test-kit on a display of the user communication device 210.

In a third step 2003, the user communication device 210 provides instructions to the user for mailing the medical sample obtained to a laboratory entity for analysis on the display of the user communication device 210. The medical sample is labeled with a sample identifier associated to the unique identifier.

In a fourth step 2004 of the method which is performed after receipt of the medical sample by the laboratory entity, the unique identifier associated with the mailed medical sample is obtained by the laboratory communication device 312, and the laboratory communication device 312 provides instructions to the laboratory entity to perform a first analysis of the medical sample.

In a fifth step 2005 which is executed after performing the first analysis, a first result of a first analysis of the medical sample is obtained by the laboratory communication device 312.

In case the first result is negative, a message indicating the negative test result is sent to the user communication device addressed by means of the unique identifier in a messaging step 2030 as indicated by arrow 2011. In case the first result is positive with respect to the predetermined medical condition, instructions associated with the unique identifier are provided to the laboratory entity or to a further laboratory entity to perform a second analysis of the medical sample using a second analysis method as indicated by arrow 2012.

A second result of the second analysis of the medical sample is obtained by the laboratory communication device 312 or a further laboratory communication device 322 in a further processing step 2020.

In case the second test result is negative with respect to the predetermined medical condition, a message indicating the negative test result is sent to the user communication device 210 addressed by means of the unique identifier in the messaging step 2030 as indicated by arrow 2021, and, if the test result is positive with respect to the predetermined medical condition, a message comprising the unique identifier is sent to a practitioner communication device 402 for establishing consultation by a medical entity, such as a medical doctor in an establishing step 2040.

While the current invention has been described in relation to its specific embodiments, it is to be understood that this description is for illustrative purposes only. The invention is not limited to the example embodiments described herein. Rather, a large number of variations are possible within the range indicated by the claims. Accordingly, it is intended that the invention be limited only by the scope of the claims appended hereto.

1-17. (canceled)

18. A method for managing information dealing with a medical test for assessing a predetermined medical condition, wherein a medical test-kit is provided, the medical test-kit comprising an access code and a sampling unit for obtaining a medical sample, the method comprising the steps of:
by a user, inputting the access code in a user communication device, wherein the access code is verified, and after verification, one of i) the access code is used as a unique identifier and ii) a unique identifier is generated, and the user communication device being configured for receiving messages addressed to the respective unique identifier;
by the user, providing a medical sample labeled with a sample identifier associated to the unique identifier to a laboratory entity for analysis, the medical sample having been obtained using components of the sampling unit; and
by the laboratory entity, receiving the medical sample and analyzing the received medical sample using a first analysis method, thereby obtaining one of a first negative test result and a first positive test result,
wherein in the case of the first negative test result, by the laboratory entity, by means of a laboratory communication device, sending a message addressed by using the unique identifier to the user communication device, the message informing the user about the first negative test result, and
wherein in the case of the first positive test result, by the laboratory entity, performing further processing, wherein the further processing comprises, by at least one of the laboratory entity and a further laboratory entity, analyzing the received medical sample using a second analysis method, thereby obtaining one of a second negative test result and a second positive test result,
wherein in the case of the second negative test result, by one of the laboratory entity and the further laboratory entity, by means of at least one of the laboratory communication device and a further laboratory communication device, sending a message addressed by using the unique identifier to the user communication device, the message informing the user about the second negative test result, and
wherein in the case of the second positive test result, establishing consultation by a medical entity, wherein the establishing comprises:
by at least one of the laboratory entity and the further laboratory entity, by means of at least one of the laboratory communication device and a further laboratory communication device, sending a message to a practitioner communication device of the medical entity, the message comprising an indication of the unique identifier, and at least one of the first positive test result and the second positive test result; and
by the medical entity, communicating with the user, wherein communication is established using the practitioner communication device which is configured to contact the user communication device identified by means of the unique identifier.

19. The method according to claim 18, wherein the user communication device, the laboratory communication device, and the practitioner communication device are configured to communicate with a central server for relaying messages between the user communication device, the laboratory communication device, and the practitioner communication device.

20. The method according to claim 18, wherein the user further provides a region code, and the medical entity is selected based on the provided region code.

21. The method according to claim 18, wherein the first analysis method detects a protein and the second analysis method detects a nucleic acid.

22. The method according to claim 18, wherein at least one of the first and second analysis methods test for the medical condition, wherein the medical condition at least one of (i) correlates with a sexually transmitted disease, and (ii) is a disease caused by one of a virus, a bacterium, a fungus and a parasite.

23. The method of claim 22, wherein the disease is caused by one of human immunodeficiency virus (HIV), hepatitis B virus (HBV), hepatitis C virus (HCV), human papillomaviruses (HPV), Chlamydia trachomatis, herpes simplex virus (HSV), Treponema pallidum, and severe acute respiratory syndrome coronavirus 2 (SARS-COV-2).

24. The method of claim 18, wherein at least one of the user communication device, the laboratory communication device, and the practitioner communication device is selected from a smartphone, a tablet and an internet enabled PC.

25. The method of claim 18, wherein the step of inputting the access code in the user communication device comprises at least one of (i) scanning of a 1D or 2D matrix code using a camera of the user communication device, and (ii) scanning of an RFID tag.

26. The method of claim 18, wherein the step of communicating with the user comprises establishing at least one of a voice link and a video link between the user communication device addressed by using the unique identifier and the practitioner communication device.

27. The method of claim 18, wherein, prior to the step of providing a medical sample, the user communication device provides detailed instructions to the user, the instructions comprising at least one of text, images, audio and video.

28. The method of claim 18, wherein, after establishing consultation by the medical entity, the method further comprises scheduling of an appointment of the user with the medical entity, wherein the unique identifier is transmitted by the user communication device to an appointment booking system of the medical entity for identification of the user.

29. The method of claim 18, wherein the method further comprises, by the laboratory entity, sending of a message addressed by using the unique identifier to the user communication device, informing the user about at least one of (i) receipt of the sample, (ii) testing of the sample, and (iii) that testing has been completed.

30. The method of claim 18, wherein the method further comprises, by the user communication device when a message indicating a negative test result has been received, the step of obtaining user input indicating whether the user belongs to a high risk group for the predetermined medical condition, wherein in the case the user belongs to a high risk group, performing at least one of:
by the user communication device, generating a reminder for the user to repeat the test after a predetermined time has passed; and
by the user communication device, offering further testing in order to assess whether the user is eligible for a prophylaxis treatment relating to the predetermined medical condition.

31. A user communication device configured for use in a method of claim 18, wherein the user communication device is configured to obtain a unique identifier by means of user input and is further configured for receiving messages from a server addressed to the unique identifier.

32. A computer program comprising instructions, which, when executed by a computer, cause the computer to carry out the steps to be executed by the user communication device of the method of claim 18, wherein the computer program includes instructions to cause the computer to:
obtain an access code by means of user input;
verify the access code;
generate a unique identifier or use the access code as unique identifier; and
receive messages addressed to the unique identifier.

33. The method of claim 18, wherein the medical test-kit further comprises a reference for a downloadable computer program to be executed on a user communication device.

34. A central server configured to carry out the steps of the central server of the method of claim 18.

35. A method for managing information dealing with a medical test for assessing a predetermined medical condition by means of a distributed system comprising a user communication device, a laboratory communication device, optionally a further laboratory communication device and a practitioner communication device, the method comprising the steps of:
obtaining by the user communication device information on an access code provided in a test-kit for assessing a predetermined medical condition, the user communication device being configured to verify the access code and for receiving and displaying messages addressed to a unique identifier, wherein at least one of the unique identifier is generated, and the access code is used as unique identifier;
providing suggestions to a user in order to assist in obtaining a medical sample using components of a sampling unit provided in the test-kit on a display of the user communication device;
providing instructions to the user for providing the medical sample obtained to a laboratory entity for analysis on the display of the user communication device, the medical sample being labeled with a sample identifier associated to the unique identifier;
by the laboratory communication device, obtaining the unique identifier associated with the provided medical sample and providing instructions to a laboratory entity to perform a first analysis of the medical sample; and
obtaining a result of the first analysis of the medical sample by the laboratory communication device, and, if the result is negative with respect to the predetermined medical condition, sending a message about the negative test result to the user communication device by means of the unique identifier, and, if the test result is positive with respect to the predetermined medical condition, providing instructions associated with the unique identifier to the laboratory entity or to a further laboratory entity to perform a second analysis of the medical sample using a second analysis method.

36. The method of claim 35, further comprising obtaining a result of the second analysis of the medical sample by at least one of the laboratory communication device and a further laboratory communication device, and, if the test result is negative with respect to the predetermined medical condition, sending a message about the negative test result to the user communication device by means of the unique identifier, and, if the test result is positive with respect to the predetermined medical condition, sending a message comprising the unique identifier to a practitioner communication device for establishing consultation by a medical entity, such as a medical doctor.
